(19) United States
(12) Patent Application Publication
(10) Pub. No.: US 2005/0159626 A1
(43) Pub. Date: Jul. 21, 2005

(54) Norbornane based cycloaliphatic compounds containing methylene amine groups

(76) Inventors: Alan Martin Allgeier, Wilmington, DE (US); Christian Peter Lenges, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/760,778

(22) Filed: Jan. 19, 2004

(51) Int. Cl. C07C 29/48

(52) U.S. Cl. 564/453; 549/297; 558/429

(57) ABSTRACT

The present invention relates to novel norbornane based cycloaliphatic methylene amine compounds, and methods for making the same using nitrile hydrogenation reactions.

### FIELD OF THE INVENTION

The present invention discloses novel norbornane-based methylene amine compounds as well as a method for making them comprising nitrile hydrogenation reactions.

### BACKGROUND OF THE INVENTION

Cycloaliphatic compounds containing methylene amine groups are of great interest as precursors to a variety of useful molecules with applications as monomers for the production of polymers, as starting materials for organic synthesis or as epoxy curing agents, either neat or as the adducted form. One skilled in the art of epoxy formulation will select different curing agents based on their structure to control curing time, pot life and physical properties of resulting coatings, adhesives, castings or composites. There is great interest in the economic preparation of cycloaliphatic amine compounds bearing different functional groups for epoxy cure applications. The methylene amine functional group can also be utilized in organic synthesis, and treatment with acids can yield ammonium salts, which may be useful as surfactants and detergents. Norbornane based methylene amine compounds are an important class of cycloaliphatic amines.

Norbornane dimethyleneamine was described in U.S. Pat. No. 3,143,570 in 1964 and its use has been reported in a variety of applications including preparation of isocyanates (JP 2764081) and in polyurethane foams (JP 2764081) since that time. Despite the utility of this amine compound little work has been invested in the modification of the norbornyl skeleton with an exception of norbornyl structures bearing a fused 5-member alicyclic ring, i.e. tricyclodecane structures derived from dicyclopentadiene (NL 64014369). GB1480999 describes the preparation and use of triamines based on the norbornane skeleton as isocyanate precursors for polyurethane lacquer formation, but fails to suggest the novel structures reported, herein.

There is a need for norbornane compounds, which contain methylene amine groups and a method to produce such norbornane derivatives, which contain methylene amine groups. These needs are met by the present invention.

### SUMMARY OF THE INVENTION

Accordingly, it is one object of the present invention to provide novel norbornane derivatives containing methylene amine groups. It is another object of the present invention to provide a method for preparing such norbornane derivatives. These and other objects will become apparent in the following detailed description.

### DETAILED DESCRIPTION OF THE INVENTION

Cycloaliphatic norbornane compounds containing methylene amine groups of formula (I) are disclosed: 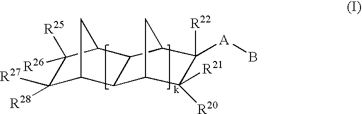
either alone, as combinations of these, and/or as mixtures of isomers of these,
wherein
k=0, 1 or 2 and the bridging CH₂ group may be on the same or opposite side with respect the first bridging CH₂ group,
   wherein
R²⁰, R²¹, R²² can be the same or different and are each independently H, a C₁ to C₂₀ alkyl group, a C₁ to C₂₀ alkyl group substituted with a hydroxyl group, a C₁ to C₁₈ perfluoroalkyl group, a phenyl group, a C₆ to C₂₀ aryl group substituted with a C, to C12 alkyl group, a C₆ to C₂₀ aryl group substituted with a hydroxyl group, a C(O)OR²⁹ group (with R²⁹ selected to be a C₁ to C₂₀ linear or branched or cyclic alkyl group or a C6 to C20 aryl group), or an alkylene chain (—(CH₂)_{q}—; q equals an integer 0-16) or nothing (in which case A or B may connect back to the norbornane skeleton), with the proviso that R²⁰, R²¹ and R²² do not comprise a cyano group or an amino group and
   wherein
A equals nothing or any alkylene chain (—(CH₂)ₚ—; p equals an integer 1-16), any substituted C₁ to C₂₀ alkylene group (provided the substituent does not comprise a cyano group or an amino group and does not interfere with the process of this invention), C₁ to C₂₀ cycloaliphatic group, a C₁ to C₂₀ hydrocarbyl or cyclohydrocarbyl group that may comprise one or more alkene groups or a C₁ to C₁₈ perfluoroalkylene group, and wherein A may form a ring of greater than 5 carbons that connects to the norbornane skeleton through R²⁰, R²¹ or R²²
   with the proviso that R²⁰, R²¹ or R²² cannot all be H if A equals nothing and
   wherein
B equals —CN, —CH₂NH₂, —(CH₂)ₛOH or —C(O)OR²⁴
   with s equal to an integer 0-12 and with R²⁴ selected to be H, a C₁ to C₂₀ linear or branched or cyclic alkyl or alkylene group, a C₆-C₂₀ aryl group or a C₁-C₁₈ perfluorinated alkyl group and wherein R²⁴ may connect to the norbornane skeleton through R²⁰, R²¹ or R²² and
      wherein
R²⁵, R²⁶, R²⁷, R²⁸ can be the same or different and are each independently H or —CH₂NH₂, with the proviso that three of R²⁵, R²⁶, R²⁷, R²⁸ are each H.
   The relative spatial orientation of the substituents on the norbornane skeleton can be any possible combination. Stereoisomeric mixtures are common embodiments of the invention.

These compounds are useful as precursors or reactants of use in various applications, for example the synthesis of complex organic molecules and use as monomers for polymeric materials.

The inventors have discovered that certain norbornane nitrile compounds (e.g. those disclosed in the inventors' concurrently filed U.S. Patent Application Ser. No. ______, filed ______ (Attorney docket no. PI-1505USNA), the disclosure of which is incorporated by reference herein in its entirety) can be contacted with hydrogen, in the presence of a catalyst and optionally a promoter at a temperature of about 60° C. to about 200° C. and a pressure of 340 kPa-17240 kPa to yield norbornane methylene amine derivatives of the formula (I), wherein the catalyst comprises a transition metal, preferably cobalt or nickel.

Thus the present invention provides a hydrogenation method for preparing norbornane derivatives, which contain methylene amine groups. Generally, the present method yields the present norbornane methylene amine derivatives as a mixture of isomers, because the starting feed may have a mixture of isomers. However, it is to be understood that both the individual compounds and also the mixtures of isomers thereof are within the scope of the present invention.

The method for making the compounds of the present invention involves a hydrogenation process of nitrile containing molecules. Thus precursor norbornane nitrile derivatives as described in the inventors' concurrently filed U.S. Patent Application Ser. No. ______, filed (Attorney docket no. PI-1505USNA) either alone or as mixtures of isomers may be contacted with hydrogen in the presence of a catalyst, optionally in the presence of a solvent to yield amine compounds described by (I).

During the hydrogenation process the feed (i.e. nitrile molecules such as those described in the inventors' concurrently filed U.S. Patent Application Ser. No. ______, filed ______ (Attorney docket no. PI-1505USNA) either alone or in mixtures of isomers) is contacted with hydrogen. The mole ratio of hydrogen to feed is not critical as long as sufficient hydrogen is present to produce the desired amines (I). Hydrogen is preferably used in excess. Hydrogen pressures are generally in the range of about 340 kPa-17240 kPa, with about 1480 to about 9000 kPa preferred. The hydrogenation process can be conducted at temperatures from 50° C. to about 180° C., preferably from 65° C. to about 100° C.

Preferred catalysts for hydrogenating nitriles to amines comprise one or more elements from the series of transition metals, particularly useful are iron, cobalt, nickel, ruthenium, rhodium and combinations thereof. The hydrogenation catalyst may also comprise one or more elements in addition to the transition metals mentioned above, for example, elements of Group IA (including lithium, sodium and potassium), elements of Group IIA (including magnesium and calcium), titanium, elements of Group VI (including chromium, molybdenum and tungsten), elements of Group VII (including palladium) and/or aluminum, silicon, boron and/or phosphorous. The hydrogenation catalyst can also be in the form of an alloy, including a solid solution of two or more elements. The hydrogenation catalyst can also be a homogeneous catalyst capable of hydrogenating nitrites to amines, e.g. HRh(PPh₃)₄ or H₂Ru(H₂)₂(PCy₃)₂.

The transition metal for hydrogenation can also be supported on an inorganic support such as alumina, magnesium oxide and combinations thereof. The metal can be supported on an inorganic support by any means known to one skilled in the art such as, for example, impregnation, co-precipitation, ion exchange, or combinations of two or more thereof. The metal can be reduced before the hydrogenation reaction by any means known to one skilled in the art such as, for example, pretreatment with hydrogen, formaldehyde or hydrazine.

The hydrogenation catalyst can be present in any appropriate physical shape or form. It can be a homogeneous catalyst, a heterogenized homogeneous catalyst or it can be in fluidizable forms, powders, extrudates, tablets, spheres or combinations of two or more thereof. The hydrogenation catalyst may be in sponge metal form, for example, the Raney® nickels and Raney® cobalts. The molar ratio of hydrogenation catalyst to feed (i.e. nitrile molecules such as those described in the previously mentioned concurrently filed U.S. Patent Application Ser. No. ______, filed ______ (Attorney docket no. PI-1505USNA) either alone or in mixtures of isomers) can be any ratio as long as the ratio can catalyze the hydrogenation. The weight ratio of hydrogenation catalyst to feed is generally in the range of from about 0.0001:1 to about 1:1, preferably about 0.001:1 to about 0.5:1. If the catalytic element is supported on an inorganic support or is a portion of an alloy or solid solution, the catalytic element is generally present in the range of from about 0.1 to about 60, preferably about 1 to about 50, and most preferably about 2 to about 50 weight percent based on the total hydrogenation catalyst weight.

The preferred nitrile hydrogenation catalyst is a sponge metal type catalyst. The metallic component is iron, cobalt, nickel or combinations thereof. Commercially available catalysts of this type are promoted or un-promoted Raney® Ni or Raney® Co catalysts that can be obtained from the W.R. Grace and Co. (Chattanooga, Tenn.), or alternative sponge metal catalysts available, for example, from Activated Metals Corporation (Sevierville, Tenn.) or Degussa (Parsippany, N.J.).

The hydrogenation can optionally be conducted in the presence of a solvent. Suitable solvents include those known in the art as useful for hydrogenation reactions. Examples of these are amines, aliphatic alcohols, aromatic compounds, ethers, esters (including lactones), and amides (including lactams). Specific examples of solvents include: ammonia, toluene, tetrahydrofuran, methanol, ethanol, any isomeric propanol, any isomeric butanol and water. Preferred solvents include ammonia and methanol. It will be appreciated that the solvent may serve to reduce the viscosity of the system to improve fluidity of the catalyst in the reaction vessel, as well as serve to remove the heat of reaction from the feed and products. The solvent may be present in a range of 1% to 75% by weight of the total reaction mixture, excluding the catalyst, preferably from 10% to 50%.

Optionally, a promoter may be used in the hydrogenation process to alter the rate of the reaction and/or to alter the selectivity of the reaction. Suitable promoters include water, alkali or alkaline earth metal hydroxides, quaternary ammonium hydroxides, quaternary ammonium cyanides, quaternary ammonium fluorides, and combinations of these. Promoters may be present at from 10 ppm to 3% by weight of the total reaction mixture, excluding the catalyst, preferably from 50 ppm to 1.5%.

It will be further appreciated that any olefin content of feed (i.e. any carbon-carbon double bonds in the structure) may be saturated using the instant hydrogenation with the further specification that the preferred catalyst for hydrogenation of the olefin comprises palladium, rhodium, nickel and/or ruthenium. Hydrogenation of the olefin content can occur before, during or after the hydrogenation of the nitrile content to amine.

The cycloaliphatic compounds used as starting material in this invention contain a nitrile substituted norbornane (bicyclo[2.2.1]heptane) fragment which is hydrogenated using the hydrogenation process of this invention to the products of this invention, the norbornane amine derivatives. The norbornane nitrile starting materials can be prepared as described in the inventors previously mentioned concurrently filed U.S. Patent Application Ser. No. ______, filed ______ (Attorney docket no. PI-1505USNA).

In a first preferred embodiment, the present invention relates to compounds with the general structure of formula (III):

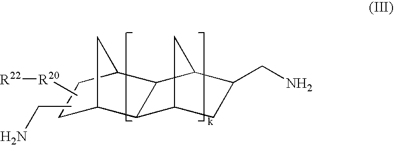

The exact point of attachment and orientation of CH₂NH₂ and R²⁰-R²² can vary and mixtures of compounds and isomers are commonly produced by this invention. Structure (III) is defined by structure (I) when A=nothing, B=CH₂NH₂, at least one of R²⁰-R²² is not H and k is defined as above.

Preferred norbornane methylene amine derivatives in this embodiment are for example structures (IV-XI): 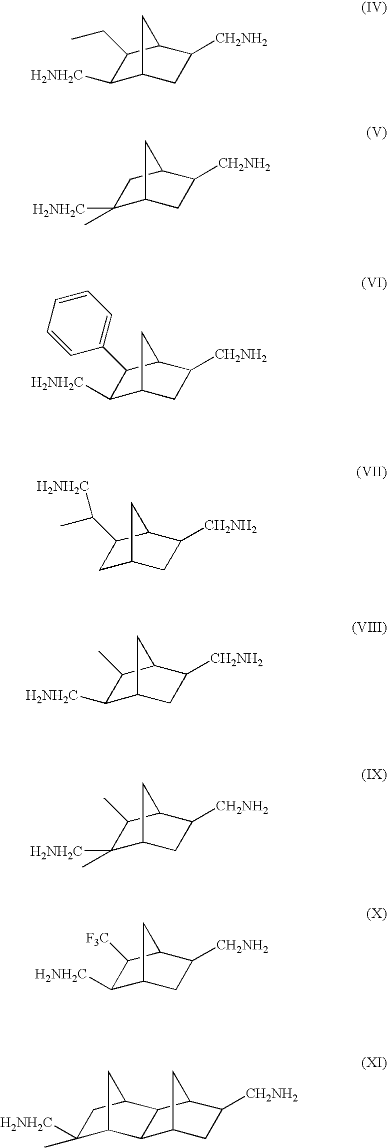
as a single isomer or as a mixture of isomers, or as a mixture of different compounds of structure (III).

For the production of the compounds of formula (IV-XI), the norbornane nitrile derivative is reacted with hydrogen in the presence of a catalyst, preferably cobalt and optionally a promoter. In this embodiment, a product mixture is obtained which generally comprises norbornane derivatives having two methylene amine groups.

In another preferred embodiment, the present invention relates to compounds with the general structure of formula (XII): 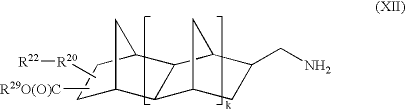
The exact point of attachment and orientation of the —C(O)OR²⁹ group and the substituents R²⁰-R²² can vary and mixtures of compounds and isomers are commonly produced by this invention. Structure (XII) is defined by structure (I) when k and R²⁹ are defined as above, A=nothing and B=C(O)OR²⁹. Preferred norbornane methylene amine derivatives in this embodiment are for example structures (XII-XVI): 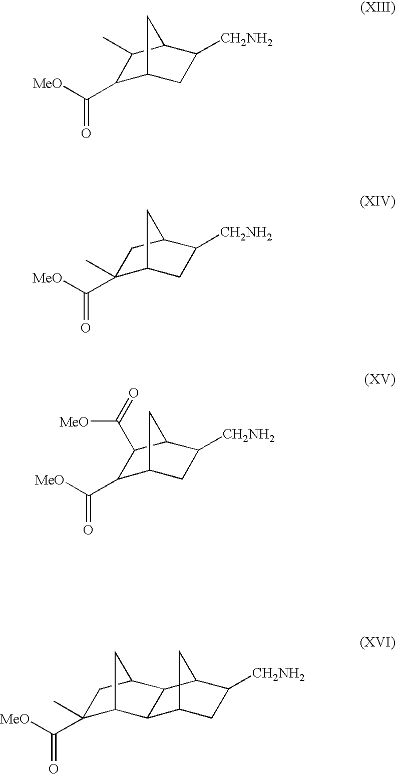
as a single isomer or as a mixture of isomers, or as a mixture of different compounds of structure (XII).

For the production of the compounds of formula (XII-XVI), the norbornane nitrile derivative is reacted with hydrogen in the presence of a group VIII catalyst, preferably cobalt and optionally a promoter. In this embodiment, a product mixture is obtained which generally comprises norbornane derivatives having one methylene amine group and one or more ester groups.

In another preferred embodiment, the present invention relates to compounds with the general structure of formula (XVII, XIX-XXI):

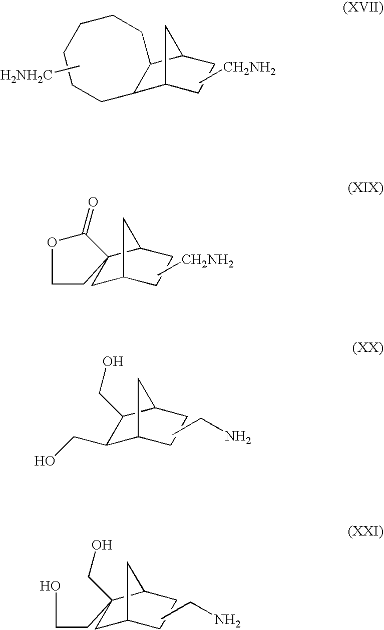

The exact point of attachment and orientation of the —CH₂NH₂ group can vary and mixtures of compounds are commonly produced by this invention.

Structure (XVII) is defined by structure (I) when k=0, and A comprises a ring that connects back to the norbornane skeleton and B equals —CH₂NH₂. Structure (XIX) is defined by structure (I) when k=0, A equals nothing, B equals C(O)OR²⁴ and R²⁴ equals —CH₂CH₂— and connects back to the norbornane skeleton. Structure (XX) is defined by structure (I) when k=0, A equals nothing, B equals CH₂OH, and R²¹ equals CH₂OH. Structure (XXI) is defined by structure (I) when k=0, A equals nothing, B equals CH₂OH, and R²¹ equals CH₂CH₂OH.

For the production of the compounds of formula (XVII, XIX-XXI), the norbornane nitrile derivative is reacted with hydrogen in the presence of a group VII catalyst, preferably cobalt, and optionally a promoter. In this embodiment, a product mixture is obtained which generally comprises norbornane derivatives having one or two methylene amine groups and in case of (XIX) a lactone group and in the case of (XX) and (XXI) alcohol groups.

It will be appreciated that the ester groups of (XII)-(XVI) and (XIX) and the anhydride group of (XVIII) may be converted to alcohol groups by methods known in the art, e.g. reduction with hydride reagents (LiAlH₄) or catalytic ester hydrogenation. Thus these compounds are intermediates to amino alcohol norbornyl compounds also of this invention.

In another preferred embodiment, the present invention relates to compounds with the general structure of formula (XXII): 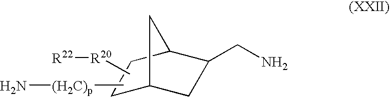
with one of the substituents R²⁰ to R²² selected independently from the group consisting of hydrogen, methyl or other branched or linear alkyl groups and with p equal to an integer 2-13.

The exact point of attachment and orientation of the —(CH₂)ₚ—NH₂ group and the substituents R²⁰—R²² can vary and mixtures of compounds and isomers are commonly produced by this invention. Structure (XXII) is defined by structure (I) when k=0, A equals (CH₂)ₚ₋₁ and B equals CH₂NH₂.

Preferred norbornane based methylene amine derivatives in this embodiment are for example structures (XXIII-XXIV):

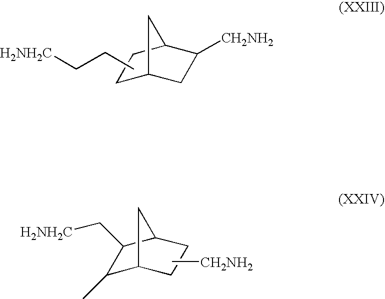

The exact point of attachment and orientation of the —CH₂NH₂ groups can vary and mixtures of compounds are commonly produced by this invention.

For the production of the compounds of formula (XXIII-XXIV) the norbornane nitrile derivative is reacted with hydrogen in the presence of a group VIII catalyst, preferably cobalt and optionally a promoter. In this embodiment, a product mixture is obtained which generally comprises norbornane derivatives having two methylene amine groups.

In another preferred embodiment, the present invention relates to compounds with the structure of formulae (XXV) and (XXVII):

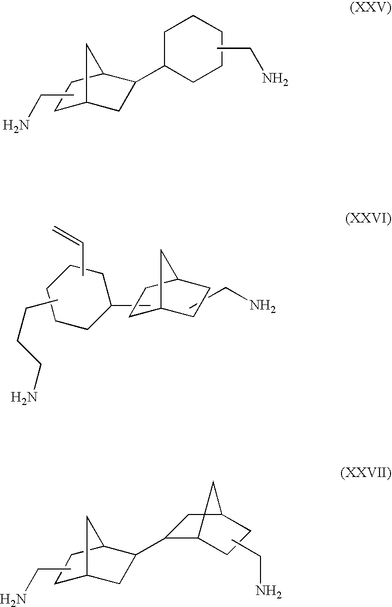

The exact point of attachment and orientation of the —CH₂NH₂ group as well as the orientation of the two cycloaliphatic rings can vary and mixtures of compounds are commonly produced by this invention.

For the production of the compounds of formula (XXV and XXVII), the norbornane nitrile derivative is reacted with hydrogen in the presence of a group VIII catalyst, preferably cobalt and optionally a promoter. In this embodiment, a product mixture is obtained which generally comprises norbornane derivatives having two methylene amine groups.

The products according to the present invention can be used as organic synthesis starting materials, monomers for the production of polymers, as epoxy curing agents or in surfactant applications.

Having generally described this invention, a further understanding can be obtained by reference to certain specific examples, which are provided herein for purpose of illustration only and are not intended to be limiting.

### EXAMPLES

### Example 1

Hydrogenation of 2-(2-cyanoethyl)-(5 or 6)-cyano-bicyclo[2.2.1]heptane (mixture of isomers). To a 100 cc pressure reactor were added 40 g of starting dinitrile, 2 g of Raney® Co 2724 slurry, and 2 g methanol (to aid in transfer). The reactor was sealed, purged with hydrogen and tested for leaks and cooled. Ammonia (17 g) was added by distillation from a cylinder. The reactor was heated to 75° C. at which point the pressure was increased to 6205 kPa (900 psig) with hydrogen and the reaction commenced. Hydrogen was constantly replenished from a 1 L pressure vessel and controlled by a forward pressure regulator. After 7.5 hours the reaction had consumed 0.93 mol H₂ from the reservoir and the reaction was cooled. An infrared spectrum of the product revealed no nitrile stretching absorbance (2238 cm⁻¹) but the presence of amine N—H stretching absorbances around 3354 cm⁻¹. The crude product was distilled under 134 mTorr vacuum and the major fraction distilled at 90° C. yielding 39.9 g colorless liquid product. NMR and IR spectra were consistent with formation of the diamine product, (XXIII).

### Example 2

Hydrogenation of 2-methyl-2, (5 or 6)dicyano-bicyclo[2.2.1]heptane (mixture of isomers). To a 4 L pressure reactor were added 900 g of starting dinitrile, 90 g of Raney® Co 2724, approximately 90 g water, and 320 g methanol (to aid in transfer). The reactor was sealed, purged with hydrogen and tested for leaks and cooled. Ammonia (600 g) was added by distillation from a cylinder. The reactor was heated to 80° C. at which point the pressure was increased to 8273 kPa (1200 psig) with hydrogen and the reaction commenced. Hydrogen was constantly replenished from a cylinder and controlled by a forward pressure regulator. After 7 hours the reaction was cooled. An infrared spectrum of the product revealed no nitrile stretching absorbance (2235 cm⁻¹) but the presence of amine N—H stretching absorbances around 3364 and 3287 cm⁻¹. NMR spectra were consistent with formation of the diamine product, (V), as well. The product was purified via distillation.

### Example 3

Hydrogenation of 1,2,3,4,4a,5,6,7,8,8a-decahydro-2-methyl-1,4:5,8-Dimethanonaphthalene-2, (6 or 7)-dicarbonitrile. To a 4 L pressure reactor were added 770 g starting dinitrile, 77 g of Raney® Co 2724, approximately 80 g water, and 700 g methanol (to aid in transfer). The reactor was sealed, purged with hydrogen and tested for leaks and cooled. Ammonia (500 g) was added by distillation from a cylinder. The reactor was heated to 80° C. at which point the pressure was increased to 8273 kPa (1200 psig) with hydrogen and the reaction commenced. Hydrogen was constantly replenished from a cylinder and controlled by a forward pressure regulator. After 7.25 hours the reaction was cooled. NMR spectra revealed the absence of any nitrile peaks in the 13C spectrum (˜125 ppm) and the formation of amine, (XI). The product was purified via distillation.

### Example 4

Hydrogenation of 1,2,3,4,4a,5,6,7,8,8a-decahydro-2-methyl-1,4:5,8-Dimethanonaphthalene-2, (6 or 7)-dicarbonitrile. To a 1 L pressure reactor were added 147 g starting dinitrile, 15 g of Raney® Co 2724, approximately 22 g water, and 200 g methanol (to aid in transfer). The reactor was sealed, purged with hydrogen and tested for leaks and cooled. Ammonia (150 g) was added by distillation from a cylinder. The reactor was heated to 85° C. at which point the pressure was increased to 8273 kPa (1200 psig) with hydrogen and the reaction commenced. Hydrogen was constantly replenished from a cylinder and controlled by a forward pressure regulator. After 4.5 hours the reaction was cooled. A gas chromatogram of the sample showed predominantly one product peak with minor side products accounting for less than 5%. An infrared spectrum of the product revealed no nitrile stretching absorbance (2235 cm⁻¹) but the presence of amine N—H stretching absorbances around 3369 and 3289 cm⁻¹. NMR spectra revealed the absence of any nitrile peaks in the 13C spectrum (˜125 ppm) and the formation of amine, (XI). The product was purified via distillation.

### Example 5

Hydrogenation of 2-ethyl-3-((5 or 6)-cyano-bicyclo[2.2.1.]hept-2-yl)-(5 or 6)-cyano-bicyclo[2.2.1]heptane (mixture of isomers). To a 4 L pressure reactor were added 550 g of the starting dinitrile, 55 g of Raney® Co 2724, approximately 50 g water, and 500 g methanol (to aid in transfer). The reactor was sealed, purged with hydrogen and tested for leaks and cooled. Ammonia (500 g) was added by distillation from a cylinder. The reactor was heated to 85° C. at which point the pressure was increased to 8273 kPa (1200 psig) with hydrogen and the reaction commenced. Hydrogen was constantly replenished from a cylinder and controlled by a forward pressure regulator. After 13 hours the reaction was cooled. An infrared spectrum of the product revealed no nitrile stretching absorbance (2235 cm⁻¹) but the presence of amine N—H stretching bands around 3350 cm⁻¹. NMR spectra were consistent with formation of the diamine product, (IV), as well. The product was purified via distillation.

### Example 6

Hydrogenation of 2-methyl-2, (5 or 6)-dicyano-bicyclo[2.2.1]heptane (mixture of isomers). To a 100 cc pressure reactor were added 15 g starting dinitrile, 2 g of Raney® Ni 2400, 4 g water, 30 g methanol and 0.06 g 50% NaOH(aq). The reactor was sealed, purged with hydrogen and tested for leaks. It was heated to 75° C. at which point the pressure was increased to 3447 kPa (500 psig) with hydrogen and the reaction commenced. Hydrogen was constantly replenished from a 1 L pressure vessel and controlled by a forward pressure regulator. After 6.5 hours the reaction had consumed 0.26 mol H₂ from the reservoir and the reaction was cooled. An infrared spectrum of the product revealed only a small nitrile stretching absorbance (2230 cm⁻¹) but the presence of amine N—H stretching absorbances around 3373 and 3294 cm⁻¹. Gas chromatography analysis of the product showed a mixture of isomers, (V), and very little remaining starting material.

### Example 7

Hydrogenation of 2-((3 or 4)-cyanocyclohex-1-yl)-(5 or 6)-cyano-bicyclo[2.2.1]heptane (mixture of isomers). To a 100 cc pressure reactor were added 20 g 2-((3 or 4)-cyanocyclohex-1-yl)-(5 or 6)-cyano-bicyclo[2.2.1]heptane, 3 g of Raney® Co 2724 slurry, and 20 g tetrahydrofuran. The reactor was sealed, purged with hydrogen and tested for leaks and cooled. Ammonia (20 g) was added by distillation from a cylinder. The reactor was heated to 75° C. at which point the pressure was increased to 6205 kPa (900 psig) with hydrogen and the reaction commenced. Hydrogen was constantly replenished from a cylinder and controlled by a forward pressure regulator. After 3 hours the reaction was cooled. An infrared spectrum of the product revealed no nitrile stretching absorbance (2235 cm⁻¹) but the presence of amine N—H stretching bands around 3365 cm⁻¹ and 3285 cm⁻¹. NMR spectra were consistent with formation of the diamine product, (XXV), as well.

### Example 8

Hydrogenation of 2-cyano-5,6-di(methoxycarbonyl)bicyclo[2.2.1]heptane. To a 100 cc pressure reactor were added 25.1 g of starting nitrile diester, 4 g of Raney® Co 2724 slurry, and 30.6 g methanol. The reactor was sealed, purged with hydrogen and tested for leaks. The reactor was heated to 70° C. at which point the pressure was increased to 3447 kPa (500 psig) with hydrogen and the reaction commenced. Hydrogen was constantly replenished from a cylinder and controlled by a forward pressure regulator. After 4 hours the reaction was stopped. An infrared spectrum of the product showed remaining nitrile so the product was charged back to the reactor with 30 g methanol and 5 g Raney® Co 2724 slurry. The reaction was carried out at 80° C. and 3447 kPa (500 psig) for 4 hours. An IR spectrum of the product showed the absence of nitrile stretching absorbance (2235 cm⁻¹) but the presence of amine N—H stretching absorbances around 3350 cm⁻¹. A gas chromatogram coupled with a mass spectrum showed a peak at m/z 241 consistent with the formation of the amine-diester (XV) and its isomers.

### Example 9

Hydrogenation of 2-(hydroxymethyl)-(5 or 6)-cyano-bicyclo[2.2.1]heptane-2-ethanol. To a 100 cc pressure reactor were added 9.0 g of starting nitrile diol, 2 g of Raney® Co 2724 slurry, and 21 g methanol. The reactor was sealed, purged with hydrogen and tested for leaks. Ammonia (20 g) was added by distillation from a cylinder. The reactor was heated to 70° C. at which point the pressure was increased to 6205 kPa (900 psig) with hydrogen and the reaction commenced. Hydrogen was constantly replenished from a cylinder and controlled by a forward pressure regulator. After 2 hours the reaction was stopped. An infrared spectrum of the product showed the absence of nitrile stretching absorbance (2235 cm⁻¹) but the presence of amine N—H and O—H stretching absorbances around 3350 cm⁻¹. A gas chromatogram coupled with a mass spectrum showed a single peak at m/z 199 consistent with the formation of the amine-diol (XXI) and its isomers.

### Examples 10-14

Methylene amines of this invention were reacted with a typical epoxy resin to prepare films. Bis(4-glycidyloxyphenyl)methane (Aldrich) was placed in a reaction vial. To this was added a di-amine of this invention in a mol ratio of 2:1 at room temperature. This mixture was mixed using a Vortex mixer for 2 minutes. The homogenous clear mixture was drawn out onto a glass plate and placed into the dry time recorder. The dry time recorder was set to a 24 hour cycle and the measurement was carried out at room temperature.

| | | BK Drying Recorder | | | | | | | |
|---|---|---|---|---|---|---|---|---|---|
| Example Number | Compound | Stage 0 | Stage 1 | | Stage 2 | | Stage 3 | | Stage 4 |
| 10 | IV | 1 hr | 1-1.5 | hr | 1.5-2.5 | hr | 2.5-5 | hr | >8 hr |
| 11 | V | 1.5 hr | 1.5-2.25 | hr | 2.25-3.25 | hr | 3.25-8 | hr | >14 hr |
| 12 | XI | 0.5 hr | 0.5-1.5 | hr | 1.5-2.25 | hr | 2.25-10 | hr | >14 hr |
| 13 | XXIII | 1 hr | 1-2 | hr | 2-3 | hr | 3-10 | hr | >12 hr |
| 14 | XXV | 1.5 hr | 1.5-3.5 | hr | 3.5-4 | hr | 4-6 | hr | >8 hr |

Stage 0: leveling,
Stage 1: basic trace,
Stage 2: film building,
Stage 3: Surface trace;
Stage 4: dry

Various modifications, alterations, additions or substitutions to the processes and compositions of this invention will be apparent to those skilled in the art without departing from the spirit and scope of this invention. This invention is not limited to the illustrative embodiments set forth herein, but rather is defined by the following claims.

1. A methylene amine composition of formula (I) or mixtures or isomers thereof: 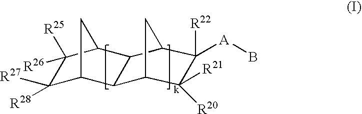
wherein
k=0, 1 or 2 and the bridging CH₂ group may be on the same or opposite side with respect the first bridging CH₂ group,
wherein
R²⁰, R²¹, R²² can be the same or different and are each independently H, a C1 to C20 alkyl group, a C1 to C20 alkyl substituted with a hydroxyl, a C1 to C18 perfluoroalkyl group, a phenyl group, a C6 to C20 aryl group substituted with a C1 to C12 alkyl, a C6 to C20 aryl group substituted with a hydroxyl, a C(O)OR²⁹ group (with R²⁹ selected to be a C1 to C20 linear or branched or cyclic alkyl group or a C6 to C20 aryl group), an alkylene chain (—(CH₂)_{q}—; q equals an integer 0-16) or nothing, and
wherein
A equals nothing or any alkylene chain (—(CH₂)ₚ—; p equals an integer 1-16), any substituted C1 to C20 alkylene group (provided the substituent does not comprise a cyano group or an amino group and does not interfere with the process of this invention), a C1 to C20 cycloaliphatic group, a C1 to C20 hydrocarbyl or cyclohydrocarbyl group that may comprise one or more alkene groups, or a C1 to C18 perfluoroalkylene group, and wherein A may form a ring of greater than 5 carbons that connects to the norbornane skeleton through R²⁰, R²¹ or R²² with the proviso that R²⁰, R²¹ or R²² cannot all be H if A equals nothing and
wherein
B equals —CN, —(CH₂)ₙOH, —CH₂NH₂ or —C(O)OR²⁴
with s equal to an integer 0-12 and with R²⁴ selected to be H, a C1 to C20 linear or branched or cyclic alkyl or alkylene group, a C6 to C20 aryl group or a C1 to C18 perfluorinated alkyl group and wherein R²⁴ may connect to the norbornane skeleton through R²⁰, R²¹ or R^{22 and}
wherein
R²⁵, R²⁶, R²⁷, R²⁸ can be the same or different and are each independently H or —CH₂NH₂, with the proviso that three of R²⁵, R²⁶, R²⁷, R²⁸ are each H.

2. The methylene amine composition according to claim 1 of structure (I) 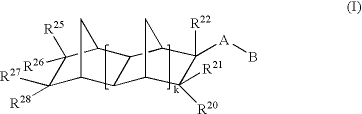
wherein
k equals 0 or 1 and A equals nothing and B is selected independently from the groups
—C(O)OR²⁹, —CN, or —CH₂NH₂
while at least one of R²⁰-R²² is selected independently from methyl, ethyl, or a C1 to C20 linear or branched alkyl group or a C1 to C18 perfluoroalkyl group or a phenyl group or a C6-C20 aryl group substituted with a C1 to C20 linear or branched alkyl group or a C6 to C20 aryl group substituted with a hydroxyl, or a —C(O)OR²⁹ group, with R²⁹ selected to be a C1 to C20 linear or branched or cyclic alkyl group or a C6 to C20 aryl group; and
one of the substituents R²⁵ to R²⁸ independently is —CH₂NH₂, while the other three substituents within the group R²⁵ to R²⁸ are hydrogen.

3. The methylene amine composition according to claim 1 of structure (I) 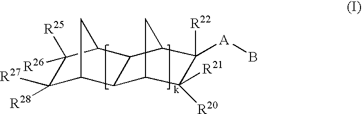
wherein k equals 0 or 1 and A equals nothing and
B plus one of the substituents R²⁰ to R²² are selected to form a lactone:
—(CH₂)ᵣ—C(O)O—(CH₂)_{q}—,
with r and q independently equal to 0, 1, 2 or higher integers, and
one of the substituents R²⁵ to R²⁸ independently is —CH₂NH₂, while the other three substituents within the group R²⁵ to R²⁸ are hydrogen.

4. The methylene amine composition according to claim 1 of structure (I) 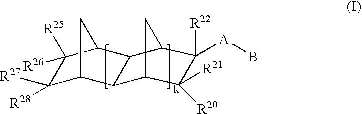
wherein k equals 0 or 1 and A equals nothing and
B equals —CH₂OH and one of R²⁰-R²² comprises a —CH₂OH or —CH₂CH₂OH and
one of the substituents R²⁵ to R²⁸ independently is —CH₂NH₂, while the other three substituents within the group R²⁵ to R²⁸ are hydrogen.

5. The methylene amine composition according to claim 1 of structure (I) 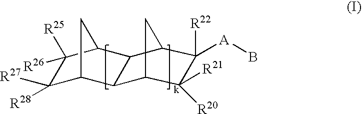
wherein k equals 0 or 1 and A and one of the substituents R²⁰ to R²² are selected to form a substituted cyclic aliphatic group,
—(CH₂)ᵣCH(B)(CH₂)_{q}—,
with r and q each equal to an integer 0-15 and wherein 2<(r+q)<15
with B equal to a cyano group (—CN) or a methylene amine —CH₂NH₂; and
one of the substituents R²⁵ to R²⁸ independently is —CH₂NH₂, while the other three substituents within the group R²⁵ to R²⁸ are each hydrogen.

6. The methylene amine composition according to claim 1 of structure (I) 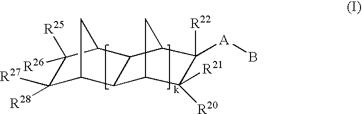
wherein
k equals 0 or 1, A equals —(CH₂)ₚ— and B equals —CN or —CH₂NH₂,
with p equal to an integer 1-12, while the substituents R²⁰ to R²² are hydrogen, methyl or a C2-C20 branched or linear alkyl group; and
one of the substituents R²⁵ to R²⁸ independently is —CH₂NH₂, while the other three substituents within the group R²⁵ to R²⁸ are each hydrogen.

7. The methylene amine composition according to claim 1 of structure (I) 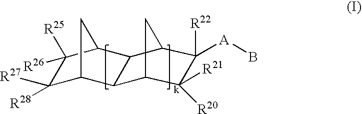
wherein
k equals 0 or 1, A is selected from
a substituted cyclohexyl group 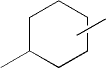
or a substituted vinyl cyclohexyl group 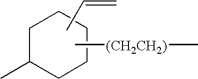
or a substituted norbornyl group 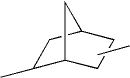
while R²⁰ to R²² are hydrogen; and
wherein B equals —CN or —CH₂NH₂; and
wherein one of the substituents R²⁵ to R²⁸ independently is —CH₂NH₂
while the other three substituents within the group R²⁵ to R²⁸ are each hydrogen.

8. A process for the preparation of substituted norbornane methylene amine compounds of claim 1 comprising contacting a corresponding substituted norbornane nitrile compound with hydrogen, in the presence of a catalyst at a temperature of about 60° C. to about 200° C. and a pressure from 340 kPa to 17240 kPa, wherein the catalyst comprises a transition metal.

9. The process of claim 8, wherein the catalyst comprises cobalt or nickel.

10. The process of claim 8 conducted in the presence of a solvent.

11. The process of claim 10, wherein the solvent comprises ammonia and or water.

12. The process of claim 8 conducted in the presence of a promoter.
